(19) United States
(12) Patent Application Publication
(10) Pub. No.: US 2007/0255072 A1
(43) Pub. Date: Nov. 1, 2007

(54) Process for the Production of Ethyl Acetate

(75) Inventors: William Fullerton, Keyingham (GB); Andrew John Miller, Hessle (GB)

Correspondence Address: NIXON & VANDERHYE, PC, 901 NORTH GLEBE ROAD, 11TH FLOOR, ARLINGTON, VA, 22203, US

(73) Assignee: BP Chemicals Limited, Middlesex (GB)

(21) Appl. No.: 11/579,135

(22) PCT Filed: May 6, 2005
(86) PCT No.: PCT/GB05/01726

§ 371 Date: Oct. 31, 2006

(30) Foreign Application Priority Data
May 12, 2004 (GB) 0410603.5

(51) Int. Cl. C07C 67/04; C07C 69/14

(52) U.S. Cl. 560/242

(57) ABSTRACT

A process for the production of ethyl acetate by reacting ethylene with acetic acid and water in the presence of a heteropolyacid catalyst in which the concentrations of reactants in the feed stream to the reactor are such that the mole ratio of ethylene to acetic acid lies in the range 6.0 to 12.2, the mole ratio of ethylene to water lies in the range 8.0 to 17.0 and the mole ratio of acetic acid to water lies in the range 1.25 to 1.40. It has been found that by careful control of the relative concentration of the reactants and of the process operating conditions the relative amounts of methyl ethyl ketone (MEK, 2-butanone) coproduced with the desired ethyl acetate can be reduced and the catalyst life can thereby be extended.

The present invention relates to a process for the synthesis of ethyl acetate by reacting an ethylene with acetic acid in the presence of an acidic catalyst.

It is well known that olefins can be reacted with lower aliphatic carboxylic acids to form the corresponding esters. One such method is described in GB-A-1259390 in which an ethylenically unsaturated compound is contacted with a liquid medium comprising a carboxylic acid and a free heteropolyacid of molybdenum or tungsten. This process is a homogeneous process in which the heteropolyacid catalyst is unsupported. A further process for producing esters is described in JP-A-05294894 in which a lower fatty acid is reacted with a lower olefin to form a lower fatty acid ester, the reaction being carried out in the gaseous phase in the presence of a catalyst consisting of at least one heteropolyacid salt of a metal e.g. Li, Cu, Mg or K, supported on a carrier. The heteropolyacid used is phosphotungstic acid and the carrier described is silica.

EP-A-0757027 (BP Chemicals) discloses a process for the production of lower aliphatic esters, for example ethyl acetate, by reacting a lower olefin with a saturated lower aliphatic carboxylic acid in the vapour phase in the presence of a heteropolyacid catalyst characterised in that an amount of water in the range from 1-10 mole % based on the total of the olefin, aliphatic mono-carboxylic acid and water is added to the reaction mixture during the reaction. The presence of water is said to reduce the amount of unwanted by-products generated by the reaction.

A general problem encountered with the above processes in the production of ethyl acetate using heteropolyacid catalysts is the generation of small amounts of a variety of by-products. These by-products generally have to be removed from the ester product by separation processes such as fractional distillation and solvent extraction. For example, the generation and recycle of acetaldehyde and methyl ethyl ketone (MEK, 2-butanone) with the feed materials can accelerate the degeneration of the catalyst and impair the quality of the product.

It has now been found that by careful control of the relative concentrations of the reactants and of the process operating conditions the relative amounts of MEK coproduced with the desired ethyl acetate can be reduced and the catalyst life can thereby be extended.

It is an object of the present invention to provide an improved process for the production of ethyl acetate by reacting ethylene with acetic acid and water in the presence of heteropolyacid catalyst. It is a further object to provide a process for the production of ethyl acetate by reacting ethylene with acetic acid and water in the presence of heteropolyacid catalyst wherein there is a reduced production of undesirable by-products.

Accordingly, the present invention is a process for the production of ethyl acetate comprising reacting ethylene with acetic acid and water in the presence of a heteropolyacid catalyst, characterised in that the concentrations of reactants in the feed stream to the reactor are such that the mole ratio of ethylene to acetic acid lies in the range 6.0 to 12.2, the mole ratio of ethylene to water lies in the range 8.0 to 17.0 and the mole ratio of acetic acid to water lies in the range 1.25 to 1.40

Preferably the concentrations of reactants in the feed stream to the reactor are such that the mole ratio of ethylene to acetic acid lies in the range 6.0 to 8.2, the mole ratio of ethylene to water lies in the range 8.0 to 11 and the mole ratio of acetic acid to water lies in the range 1.25 to 1.30.

The term “heteropolyacid” as used herein and throughout the specification is meant to include the free acids and/or metal salts thereof. The heteropolyacids used to prepare the esterification catalysts of the present invention therefore include inter alia the free acids and co-ordination type salts thereof in which the anion is a complex, high molecular weight entity. The heteropolyacid anion comprises from two to eighteen oxygen-linked polyvalent metal atoms, which are generally known as the “peripheral” atoms. These peripheral atoms surround one or more central atoms in a symmetrical manner. The peripheral atoms are usually one or more of molybdenum, tungsten, vanadium, niobium, tantalum and other metals. The central atoms are usually silicon or phosphorus but can comprise any one of a large variety of atoms from Groups I-VIII in the Periodic Table of elements. These include, for instance, cupric ions; divalent beryllium, zinc, cobalt or nickel ions; trivalent boron, alulllinium, gallium, iron, cerium, arsenic, antimony, phosphorus, bismuth, chromium or rhodium ions; tetravalent silicon, germianium, tin, titanium, zirconium, vanadium, sulphur, tellurium, manganese nickel, platinum, thorium, hafnium, cerium ions and other rare earth ions; pentavalent phosphorus, arsenic, vanadium, antimony ions; hexavalent tellurium ions; and heptavalent iodine ions. Such heteropolyacids are also known as “polyoxoanions”, “polyoxometallates” or “metal oxide clusters”.

Heteropolyacids usually have a high molecular weight e.g. in the range from 700-8500 and include dimeric complexes. They have a relatively high solubility in polar solvents such as water or other oxygenated solvents, especially if they are free acids and in the case of several salts, and their solubility can be controlled by choosing the appropriate counter-ions. Specific examples of heteropolyacids and their salts that may be used as the catalysts in the present invention include:

| | |
|---|---|
| 12-tungstophosphoric acid | H₃[PW₁₂O₄₀]•xH₂O |
| 12-molybdophosphoric acid | H₃[PMo₁₂O₄₀]•xH₂O |
| 12-tungstosilicic acid | H₄[SiW₁₂O₄₀]•xH₂O |
| 12-molybdosilicic acid | H₄[SiMo₁₂O₄₀]•xH₂O |
| Cesium hydrogen tungstosilicate | Cs₃H[SiW₁₂O₄₀]•xH₂O |
| Potassium tungstophosphate | K₆[P₂W₁₈O₆₂]•xH₂O |
| Ammonium molybdodiphosphate | (NH₄)₆[P₂Mo₁₈O₆₂]•xH₂O |

Preferred heteropolyacid catalysts for use in the present invention are tungstosilicic acid and tungstophosphoric acid. Particularly preferred are the Keggin or Wells-Dawson or Anderson-Evans-Perloff primary structures of tungstosilicic acid and tungstophosphoric acid.

The heteropolyacid catalyst whether used as a free acid or as a salt thereof can be supported or unsupported. Preferably the heteropolyacid is supported. Examples of suitable supports are relatively inert minerals with either acidic or neutral characteristics, for example, silicas, clays, zeolites, ion exchange resins and active carbon supports. Silica is a particularly preferred support. When a support is employed, it is preferably in a form which permits easy access of the reactants to the support. The support, if employed, can be, for example, granular, pelletised, extruded or in another suitable shaped physical form. The support suitably has a pore volume in the range from 0.3-1.8 ml/g, preferably from 0.6-1.2 ml/g and an average single pellet crush strength of at least 7 Newton force. The crush strengths quoted are based on average of that determined for each set of 50 particles on a CHATTILLON tester which measures the minimum force necessary to crush a single particle between parallel plates. The support suitably has an average pore radius (prior to supporting the catalyst thereon) of 10 to 500 Å preferably an average pore radius of 30 to 150 Å.

In order to achieve optimum performance, the support is suitably free from extraneous metals or elements which can adversely affect the catalytic activity of the system. If silica is employed as the sole support material it preferably has a purity of at least 99% w/w, i.e. the impurities are less than 1% w/w, preferably less than 0.60% w/w and more preferably less than 0.30% w/w.

Preferably the support is derived from natural or synthetic amorphous silica. Suitable types of silica can be manufactured, for example, by a gas phase reaction, (e.g. vaporisation of SiO₂ in an electric arc, oxidation of gaseous SiC, or flame hydrolysis of SiH₄ or SiCl₄), by precipitation from aqueous silicate solutions, or by gelling of silicic acid colloids. Preferably the support has an average particle diameter of 2 to 10 mm, preferably 4 to 6 mm. Examples of commercially available silica supports that can be employed in the process of the present invention are Grace 57 granular and Grace SMR 0-57-015 extrudate grades of silica. Grace 57 silica has an average pore volume of about 1.15 ml/g and an average particle size ranging from about 3.0-6.0 mm.

The impregnated support can be prepared by dissolving the heteropolyacid, in e.g. distilled water, demineralised water, alcohols such as methanol, ethanol, propanol, butanols and other suitable non-aqueous solutions and then adding the aqueous Solution so formed to the support. The support is suitably left to soak in the acid solution for a duration of up to several hours, with periodic manual stirring, after which time it is suitably filtered using a Buchner funnel in order to remove any excess acid.

The wet catalyst thus formed is then suitably placed in an oven at elevated temperature for several hours to dry, after which time it is allowed to cool to ambient temperature in a desiccator. The weight of the catalyst on drying the weight of the support used and the weight of the acid on support were obtained by deducting the latter from the former from which the catalyst loading in g/litre was determined.

Alternatively, the support may be impregnated with the catalyst using by spraying a solution of the heteropolyacid on to the support with simultaneous or subsequent drying (e.g. in a rotary evaporator). The support may be impregnated in commercial quantities by employing equipment of suitable scale, using procedures analogous to those described above or by any other well known method of absorbent support impregnation.

This supported catalyst can then be used in the esterification process. The amount of heteropolyacid deposited/impregnated on the support for use in the esterifcation reaction is suitably in the range from 10 to 60% by weight, preferably from 30 to 50% by weight based on the total weight of the heteropolyacid and the support.

The source of the ethylene reactant used in the present invention may be a refinery product or a chemical or a polymer grade of ethylene which may contain some alkanes admixed therewith.

Preferably the reactants fed or recycled to the reactor contain less than 1 ppm, most preferably less than 0.1 ppm of metals, or metallic compound or basic nitrogen (e.g. ammonia or amine) impurities. Such impurities can build up in the catalyst and cause deactivation thereof.

The reaction is preferably carried out in the vapour phase at a temperature suitably above the dew point of the reactor contents comprising the reactant acid, any alcohol formed in situ, and the produced ethyl acetate. The meaning of the term “dew point” is well known in the art, and is essentially, the highest temperature for a given composition, at a given pressure, at which liquid can still exist in the mixture. The dew point of any vaporous sample will thus depend upon its composition.

The supported heteropolyacid catalyst is suitably used as a fixed bed which may be in the form of a packed column, or radial bed or a similar commercially available reactor design. The vapours of the reactant olefins and acids are passed over the catalyst suitably at a GHSV in the range from 100 to 5000 per hour, preferably from 300 to 2000 per hour.

The reaction is suitably carried out at a temperature in the range from 150-200° C., preferably 160 to 195° C.

The reaction pressure is suitably in the range 8 to 20 barg (800 to 2000 KPa), preferably in the range 11 to 20 barg, more preferably from 12 to 15 barg (1200 to 1500 Kpa).

Advantages which can be obtained by the use of the process of the present invention are (1) undesirable by products such as 2-butanone and acetaldehyde may be controlled by careful adjustment of feed composition and reaction temperatures while maintaining acceptable ethyl acetate yields, (2) the production of C₄ unsaturated hydrocarbons is significantly reduced (3) the catalyst lifetime may be significantly extended (4) the process economics are improved by a reduced requirement to operate process purge streams to reduce the recycle of undesirable by-products and by the ability to de-bottleneck the product purification system.

The invention is now illustrated in the following Examples and the accompanying drawings.

### EXAMPLE

The Example was performed in a demonstration plant incorporating feed, reaction and product recovery sections, including recycle of the major by-product streams and known as a “fully recycling pilot plant”. An outline description of the layout and mode of operation of this equipment is given below.

Catalyst productivity towards some components is reported in STY units, (defined as grams of quoted component produced per litre of catalyst per hour). The apparatus used to generate this Example was an integrated recycle pilot plant designed to mimic the operation of a 220kte commercial plant at an approximate scale of 1:7000.

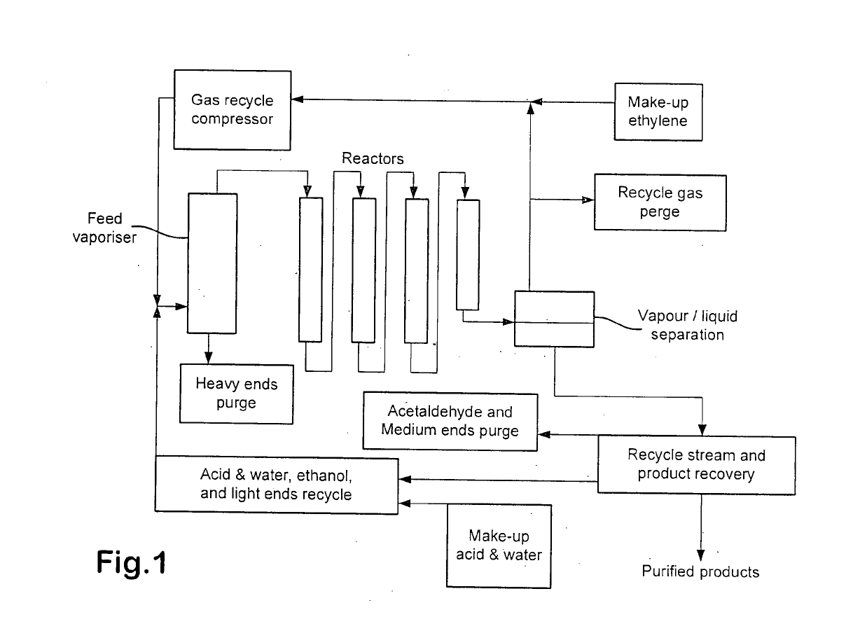

A basic flow diagram of the unit is shown in FIG. 1 of the Drawings. The unit comprises a feed section (incorporating a recycle system for both unreacted feeds and all the major by-products), a reaction section, and a product and by-product separation section. The feed section utilises liquid feed pumps to deliver fresh acetic acid, fresh water, unreacted acid/water, ethanol and light ends recycle streams to a vaporiser. The ethylene feed also enters the vaporiser where it is premixed with the liquid feeds. The ethylene is fed both as a make-up stream, but more predominantly as a recycle stream and is circulated around the system at a desired rate and ethylene content. The combined feed vapour stream is fed to a reactor train; comprising four fixed bed reactors, each containing a 5 litre catalyst charge.

The first three reactors are fitted with acid/water injection to the exit streams to both facilitate independent control of reactor inlet temperatures and to maintain the desired ethylene: acid ratio.

The crude product stream exiting the reactors is cooled before entering a flash vessel where the separation of non-condensable (gas) and condensable (liquid) phases occurs. The recovered gas is recycled back to the vaporiser with the exception of small bleed stream removed to assist control of recycle stream purity. The liquid stream enters the product separation and purification system, which is a series of distillation columns designed to recover and purify the final product and also to recover the unreacted acetic acid, water, ethanol and light ends streams for recycling back to the vaporiser. Small bleed streams located in the liquid recovery enable the removal of undesired recycle components from the process during this stage.

Analysis and Reporting

The sample points for analysis in the Example was as follows; the ethyl acetate production reported is recorded at point (a) and calculated using Coriolis meter mass flow measurement and Near Infrared (NIR) analysis of the crude liquid stream composition, calibrated in wt %.

The reported figures for MEK and acetaldehyde production are recorded on the residual crude product after the acid/water recycle stream has been separated. The stream composition is measured using an Agilent model 6890 gas liquid chromatograph equipped with both FID and TCD detectors to determine both major (wt%) and minor (ppm) components. The fitted column is a 60 m×0.32 mm i.d. DB1701 with a 1 μm film thickness operated on helium carrier gas flow of 2 ml min⁻¹ and split ratio of 25:1. The sampling system employed is an online closed loop system, with continuous sample flushing.

Experimental Conditions

The catalyst employed was 12-tungstosilicic heteropolyacid supported on Grace 57 silica with a catalyst loading of 140 grams per litre.

The experiment involved start-up and initial operation within standard parameters, described herein as feed 1, until stable baseline activity and impurity make rates were obtained. The reactor feed conditions were then altered by adjusting recycle compressor and pump flow rates. The reaction temperature was increased to maintain the catalyst productivity of ethyl acetate. The process variable alterations were made in parallel, but incrementally to avoid excessive process upset. A summary of the key process variables and experimental data obtained is given in Tables 1 and 2.

TABLE 1
Experimental conditions

| | | Feed 1 | Feed 2 | Feed 3 |
|---|---|---|---|---|
| Reaction pressure | Bar (abs) | 12 | 12 | 12 |
| Ethylene:acetic acid | Mol %/Mol % | 12.2:1 | 8.2:1 | 6.6:1 |
| Ethylene:water | Mol %/Mol % | 17:1 | 11.0:1 | 8.5:1 |
| Acetic acid:water | Mol %/Mol % | 1.40:1 | 1.33:1 | 1.29:1 |
| Recycle gas rate | kg/hr | 26.0 | 21.0 | 17.2 |
| Recycle gas purity | % v/v C2- | 90.0 | 90.0 | 90.0 |
| Reactor inlet | ° C. | 175 | 178 | 182 |
| temperature | | | | |
| (averaged) | | | | |
| Flash separation | ° C. | 30 | 30 | 30 |
| temperature | | | | |

TABLE 2
Experimental results

| Product/Impurities | | Feed 1 | Feed 2 | Feed 3 |
|---|---|---|---|---|
| Ethyl acetate STY | g/litre cat/hr | 200 | 200 | 200 |
| 2-butanone | ppm | 43 | 27 | 12 |
| Acetaldehyde | ppm | 200 | 132 | 60 |
| Diethylether | ppm | 20365 | 18640 | 11800 |
| C4 Butene species | ppm | 520 | 317 | 125 |
| (total) | | | | |
| Hexane | ppm | 21 | 21 | 16 |

As can be noted from Table 1, the effect of decreasing ethylene to water ratio over the experimental range requires increased reactor inlet temperatures to maintain a steady ethyl acetate STY. From Table 2, it is shown that, even at these elevated temperatures, the catalyst selectivity is improved, on moving firstly from feed 1 to feed 2 and then to feed 3 compositions. This is clearly illustrated in the given examples by significant reductions to 2-butanone, acetaldehyde, and diethylether production. Similar reduction trends are also observed for C₄ and the attendant derivative C₆ to C₂₀ hydrocarbon species as illustrated by hexane in the Example.

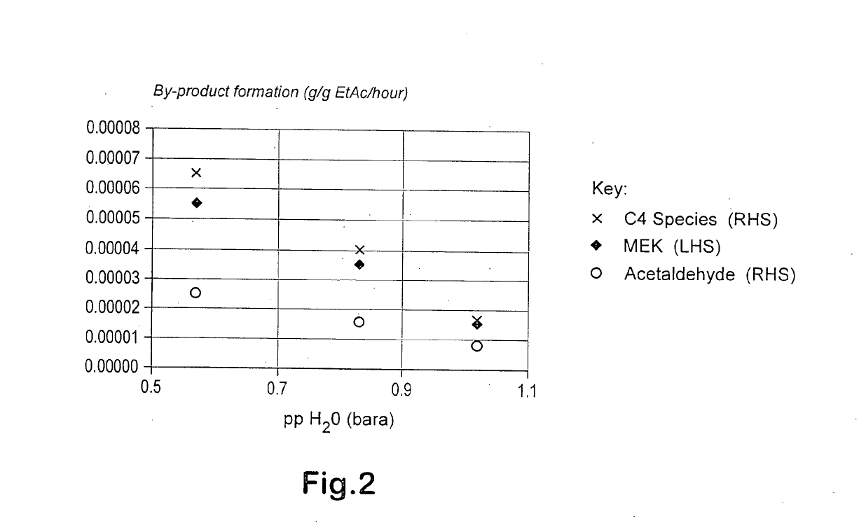

This increased selectivity may also be represented as a function of water partial pressure in FIG. 2.

The reductions in acetaldehyde and 2-butanone for example enable extended catalyst life as these materials have previously been identified as playing a role in catalyst deactivation. The broad reduction in derivative hydrocarbon species will also confer prolonged catalyst life by removing a source of coking materials for the catalyst surface that would otherwise form a barrier between the reactants and the catalyst active sites. Further economic benefit is realised by optimising feed composition to enable the reduction or elimination of various process purge streams which may be otherwise employed to prevent recycle of components detrimental to catalyst life, as otherwise valuable recyclable materials and feedstock are also inevitably removed along with the undesirable components. A further advantage is given by the reduced requirement to remove these impurities, thereby allowing an effective de-bottleneck of the process product purification system.

1-32. (canceled)

33. A process for the production of ethyl acetate comprising reacting ethylene with acetic acid and water in the presence of a heteropolyacid catalyst, characterised in that the concentrations of reactants in the feed stream to the reactor are such that the mole ratio of ethylene to acetic acid lies in the range 6.0 to 12.2, the mole ratio of ethylene to water lies in the range 8.0 to 11 and the mole ratio of acetic acid to water lies in the range 1.25 to 1.40

34. A process according to claim 33, wherein the mole ratio of ethylene to acetic acid lies in the range 6.0 to 8.2.

35. A process according to claim 33 or claim 34, wherein the mole ratio of acetic acid to water lies in the range 1.25 to 1.30.

36. A process according to claim 33 wherein the mole ratio of ethylene to acetic acid lies in the range 6.0 to 8.2, the mole ratio of ethylene to water lies in the range 8.0 to 11 and the mole ratio of acetic acid to water lies in the range 1.25 to 1.30.

37. A process according to claim 33 or claim 34 wherein the heteropolyacid catalyst is selected from a tungstosilicic acid, a tungstophosphoric acid or salts thereof.

38. A process according to claim 33 or claim 34 wherein the heteropolyacid catalyst is supported.

39. A process according to claim 38 wherein the support is selected from the group consisting of a silica, clays, zeloites, ion exchange resins, active carbons and mixtures thereof.

40. A process according to claim 39 wherein the support is a silica.

41. A process according to claim 40 wherein the silica is derived from natural or synthetic amorphous silica.

42. A process according to claim 40 wherein the silica has a purity of at least 99% by weight.

43. A process according to claim 38 wherein the support has a pore volume in the range from 0.3 to 1.8 ml/g.

44. A process according to claim 38 wherein the support has an average single pellet crush strength of at least 7 Newton force.

45. A process according to claim 38 wherein the support has an average pore radius of 10 to 500 Angstroms.

46. A process according to claim 45 wherein the support has an average pore radius of 30 to 150 Å.

47. A process according to claim 38 wherein the support has an average particle diameter of 2 to 10 mm.

48. A process according to claim 47 wherein the support has an average particle diameter of 4 to 6 mm.

49. A process according to claim 40 wherein the silica has an average pore volume of about 1.15 ml/g and an average particle size in the range about 3 to 6 mm.

50. A process according to claim 38 wherein the amount of heteropolyacid catalyst on the support is between 10 and 60% by weight.

51. A process according to claim 50 wherein the amount of heteropolyacid catalyst on the support is between 30 and 50% by weight.

52. A process according to claim 33 or claim 34 wherein the reactants contain less than 1 ppm of metals, metallic compounds or basic nitrogen impurities.

53. A process according to claim 52 wherein the amount of impurities is less than 0.1 ppm.

54. A process according to claim 33 and 34 wherein the process is carried out in the vapour phase.

55. A process according to claim 54 wherein the reaction is carried out above the dew point of the reactor contents.

56. A process according to claim 33 or claim 34 wherein ethylene and acetic acid vapours are passed over the catalyst at a GHSV of 100 to 5000 per hour.

57. A process according to claim 56 wherein the GHSV is 300 to 2000 per hour.

58. A process according to claim 33 or claim 34 wherein the reaction is carried out at a temperature in the range from 150 to 200° C.

59. A process according to claim 58 wherein the reaction is carried out at a temperature in the range from 160 to 195° C.

60. A process according to claim 33 or claim 34 wherein the reaction pressure is in the range 8 to 20 barg.

61. A process according to claim 60 wherein the reaction pressure is in the range 11 to 20 barg.

62. A process according to claim 61 wherein the reaction pressure is in the range 12 to 15 barg.

63. A process according to claim 33 or claim 34 wherein the heteropolyacid catalyst is a tungstosilicic heteropolyacid and which is supported on silica.
